(19) United States
(12) Patent Application Publication
(10) Pub. No.: US 2005/0287083 A1
(43) Pub. Date: Dec. 29, 2005

(54) Three-phase styling product

(76) Inventors: Winfried Emmerling, Tornesch (DE); Anja Thammasiri, Hamburg (DE); Uwe Bergemann, Hamburg (DE)

Correspondence Address: DANN DORFMAN HERRELL AND SKILLMAN, Suite 2400, 1601 Market Street, Philadelphia, PA, 19103-2307, US

(21) Appl. No.: 11/184,610

(22) Filed: Jul. 19, 2005

(30) Foreign Application Priority Data
Jan. 20, 2003 (DE) 103 02 190.6

(51) Int. Cl. A61K 9/00; A61K 7/00

(52) U.S. Cl. 424/47

(57) ABSTRACT

A sprayable agent for hair treatment, which is optically appealing by its three-phase formation and whose cosmetic activity can be even better visualized by the consumer is comprised of (A) an aqueous phase, (B) an oil phase and (C) a phase comprising a trifunctional ester wherein the composition is a stable, three-phase system.

### CROSS-REFERENCE TO RELATED APPLICATIONS

This application is a continuation under 35 U.S.C. § 365(c) and 35 U.S.C. § 120 of international application PCT/EP2003/014761, filed Dec. 23, 2003. This application also claims priority under 35 U.S.C. § 119 of DE 103 02 190.6, filed Jan. 20, 2003, which is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The invention relates to stable, three-phase agents for treating hair, comprising three mutually immiscible phases with a content of an aqueous phase, a water-insoluble oily body and a liquid ester.

Nowadays, many treatments are carried out on keratinic fibers, especially human hair. Treatments, which provide a permanent or temporary hairstyling, play an important role here. Temporary styling intended to provide a good set, without compromising the healthy appearance of the hair, such as, for example the gloss, can be obtained by the use of hairsprays, hair waxes, curlers, aerosols etc.

Products based on film builders, plasticizers and synthetic oils are especially widely used in the field of styling of human hair. In hairstyling, for example, they form the basis for gloss-providing hair sprays, wherein the film builder provides a hair setting and the oil the gloss. The plasticizers provide the plasticizing effect of the film builders.

Normally, pumpable hair sprays and pumpable setting sprays are offered as one-phase products in opaque packaging. Recently, however, two-phase products in transparent packaging have been increasingly manufactured, which make it easy for the consumer to see the liquid level and provides a more attractive product.

EP 1 169 998 A1 discloses transparent aerosol packaging with a two-phase agent that consists of two distinct, liquid phases separated by a sharp phase interface. The hydrophilic phase contains a cationic hair care material and the hydrophobic phase contains a liquefied propellant.

DE 197 03 475 A1 teaches two-phase, sprayable hair care agents, which comprise cationic or nonionic as well as amphoteric surfactants and an alcohol in a hydrophilic phase, and a volatile silicon compound and a non-volatile silicon compound in a lipophilic phase.

However, two-phase products contain a plurality of different active principles for styling, care and protection of the hair, which are not visible to the consumer, and therefore it is a problem to convince the consumer of the improved performance.

On the other hand, the formation of a third liquid phase by adding additional cosmetic components is not known up to now, as they either dissolve in one or the other phase or because they are emulsifiers that form a three-phase emulsion out of both phases. Indeed, attempts have recently been made to develop three-phase systems with good stability for cosmetic applications; however, these systems require a high salt content, which can lead to undesirable residues when applied to the hair.

The objective was therefore to develop a sprayable agent for hair treatment, which is optically appealing by its three-phase formation and whose cosmetic activity can be even better visualized by the consumer.

WO 92/13511 A1 discloses three-phase systems having a liquid, water-insoluble oil, a special emulsifier and an aqueous phase. These three-phase systems are intended to be suitable for tertiary oil delivery and for pharmaceutical or cosmetic products.

Surprisingly, it has now been discovered that by adding a specific plasticizer to a cosmetic, sprayable agent comprising an aqueous phase and an oil phase, a third liquid phase can be formed. The cosmetic active principles can thereby be dispersed between three phases, allowing the consumer to better visualize the effectiveness, optionally by additional coloration of the phases.

The improved visualization also enhances the belief in a complex agent having improved performance.

The three phases are stable and can be mixed in such a way that all three components arrive simultaneously at the pumping operation for application and afterwards separate out again into three phases.

(2) Description of Related Art including information disclosed under 37 CFR 1.97 and 1.98. Not Applicable.

### BRIEF SUMMARY OF THE INVENTION

Accordingly, the subject of the invention is stable, three-phase, sprayable hair treatment agents comprising three mutually immiscible liquid phases, composed of
(A) an aqueous phase;
(B) an oil phase; and
(C) a phase comprising a trifunctional ester.

### BRIEF DESCRIPTION OF THE SEVERAL VIEWS OF THE DRAWING(S)

Not Applicable

### DETAILED DESCRIPTION OF THE INVENTION

According to the invention, esters are preferred that are composed of both units a) and b), wherein
(a) unit a) stands for alcohols or carboxylic acids with chain lengths of 2 to 10 carbon atoms and
(b) unit b) stands for compounds of Formula (I), 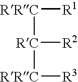
   wherein each of R¹, R² and R³ is independently an —OH or —COOH group and each of R, R″ is independently —H, —OH or a Ci-C4-alkyl group.

According to the invention, trifunctional esters are understood to mean those esters in which each of R¹, R² and R³ of Formula (I) has been esterified with alcohols and/or carboxylic acids having chain lengths of 2 to 10 carbon atoms.

Preferred compounds of Formula (I) are those in which each of R¹, R² and R³ is a hydroxy group. glycerol-is particularly preferred.

Particularly preferred esters in the context of the invention are esters, which consist of glycerol and carboxylic acids having 2 to 6 carbon atoms.

In the context of the invention, a quite particularly preferred ester is glycerol triacetate.

Further preferred compounds of Formula (I) are those in which the radicals R stand for —COOH groups, citric acid being particularly preferred.

Particularly preferred esters in the context of the invention are also those, which consist of citric acid and alcohols having 2 to 6 carbon atoms.

In the context of the invention, a quite particularly preferred ester is triethyl citrate.

According to the invention, a preferred ester content is 2 to 98 wt. %, based on the total weight of the agent, particularly 10 to 50 wt. %.

The second obligatory component of the invention is the oil component, which is responsible for producing the gloss on the hair during the hair treatment.

In principle, the oil component can be selected from both vegetal and mineral or synthetic oils, as well as mixtures of these components.

Normally, triglycerides and mixtures of triglycerides are used as the natural (vegetal) oils. In the context of the invention, preferred natural oils are coconut oil, (sweet) almond oil, walnut oil, pear kernel oil, avocado oil, tea tree oil, soybean oil, sesame oil, sunflower oil, camellia oil, evening primrose oil, rice grain oil, palm kernel oil, mango kernel oil, cuckoo flower oil, safflower oil, macadamian nut oil, grape seed oil, apricot kernel oil, olive oil, wheat germ oil, pumpkin seed oil, abutilon seed oil, hazelnut oil, Carthamus tinctorius oil, rapeseed oil, winter rose oil, jojoba oil and shea butter.

According to the invention, particularly preferred are (sweet) almond oil, avocado oil, soybean oil, sesame oil, sunflower oil, palm kernel oil, mango kernel oil, macadamia nut oil, apricot kernel oil, olive oil, wheat germ oil, pumpkin seed oil, abutilon seed oil, hazelnut oil and jojoba oil.

Mineral oils, paraffin oils and isoparaffin oils as well as synthetic hydrocarbons are preferably used as the mineral oils. A hydrocarbon that can be used according to the invention is, for example, the commercially available product 1,3-di(2-ethylhexyl)cyclohexane (Cetiol® S).

According to the invention, preferred mineral oils are paraffin oils.

Silicon compounds, especially dialkyl and alkylarylsilicones, such as, for example dimethylpolysiloxane and methylphenylpolysiloxane, as well as their hydroxyl terminated, alkoxylated and quaternized analogs come into consideration as synthetic oils. Exemplary silicones are the products marketed by Dow Corning under the trade names DC 190, DC 200, DC 344 and DC 345 (cyclomethicone).

According to the invention, preferred silicon compounds are linear and cyclic, non-alkoxylated dialkylsiloxanes and alkylarylsiloxanes.

In addition, inventive usable oils include dialkyl ethers.

Dialkyl ethers suitable for use in accordance with invention are particularly di-n-alkyl ethers containing a total of 12 to 36 carbon atoms and, more particularly 12 to 24 carbon atoms such as, for example, di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether and di-tert.butyl ether, diisopentyl ether, di-3-ethyldecyl ether, tert.butyl n-octyl ether, isopentyl n-octyl ether and 2-methylpentyl n-octyl ether.

Di-n-octyl ether, obtainable as the commercial product Cetiol® OE, is particularly preferred according to the invention.

Although oils that are usually liquid at room temperature, i.e., 25° C., are added, the invention, however, also includes mixtures of liquid and solid oil components, in so far as these mixtures are liquid at room temperature.

In a further preferred embodiment of the invention, the phase (B) includes liquid, halogenated hydrocarbons as the third phase, instead of the oil components. These can be chosen among customary chlorinated and/or fluorinated hydrocarbons. Ethyl perfluorobutyl ether and/or perfluorodecahydronaphthalene are particularly suitable in the context of the invention.

The agents according to the invention preferably comprise the oil component or the mixture of oil components or the liquid, halogenated hydrocarbons in quantities of 2 to 98 wt. %, particularly in quantities of 5 wt. % to 50 wt. %, based on the total weight of the agent.

Oil component(s) in quantities in the range of 10 to 40 wt. %, based on the total weight of the agent, are particularly preferred.

According to the invention, the aqueous phase comprises a proportion of 2 to 98 wt. %, particularly 10 to 50 wt. %, based on the total weight of the agent.

The total system can also comprise alcohols such as ethanol or isopropanol. According to the invention, the alcohol content comprises 0-30 wt. %, preferably 0-20 wt. % and particularly 0-10 wt. %, in each case based on the total weight of the agent.

In a further preferred embodiment of the invention, the agents additionally comprise at least one water-soluble film builder, which is normally a polymer.

Preferred water-soluble polymers can be of nonionogenic, amphoteric, zwitterionic or anionic nature.

According to the invention, exemplary nonionogenic polymers can be the following compounds:
polyvinyl pyrrolidones, such as, for example, those marketed by BASF under the trade name Luviskol®), for example Luviskol® K 30,
vinyl pyrrolidone-vinyl acetate copolymers, such as, for example, those marketed by BASF under the trade name Luviskol®, Luviskol® VA 64 and Luviskol® VA 73,
cellulose ethers, such as hydroxypropyl cellulose, hydroxyethyl cellulose and methylhydroxypropyl cellulose, such as, for example, those marketed by Aqualon under the trade names Cumlina® and Benecel®.

Particularly preferred water-soluble nonionogenic polymers in the context of the invention are the products marketed under the trade names Luviskol® K 30, Luviskol® VA 64 and Luviskol® VA 73.

Suitable amphoteric polymers according to the invention are, for example, the octylacrylamide-methyl methacrylate-tert.-butylaminoethyl methacrylate-2-hydroxypropyl methacrylate copolymers available under the designations Amphomer® and Amphomer® LV-71 (DELFT NATIONAL).

Suitable anionic polymers according to the invention are inter alia:
vinyl acetate-crotonic acid copolymers, such as, for example, commercialized under the designations Resyn® from National Starch Co., Luviset® from BASF and Gafset® from GAF,
vinyl pyrrolidone-vinyl acrylate copolymers, available for example from BASF under the trade name Luviflex®. A preferred example of this product is vinyl pyrrolidone—acrylate terpolymers, available under the trade name Luviflex® VBM 35 from BASF.
acrylic acid—ethyl acrylate—N-tert.-butylacrylamide terpolymers, marketed for example under the designation Ultrahold® strong, from BASF.

The water-soluble film builders can be added to the inventive agents in quantities of 0.5 to 15 wt. %, particularly 2 to 8 wt. %, based on the total weight of the agent.

In a further preferred embodiment of the invention, the agents additionally comprise oil-soluble and water-soluble hair-care materials, oil-soluble and water-soluble UV-absorbers, perfume oils and/or colorants.

According to the invention, oil-soluble and water-soluble hair care materials are understood to mean cationic orcationically active hair care materials, such as cationic polymers, cationic surfactants or cationically derivatized protein hydrolyzates, vitamins and vitamin derivatives as well as oil-soluble waxes.

Exemplary suitable cationic polymers are cationic cellulose derivatives, such as, e.g., a quaternized hydroxyethyl cellulose, available under the trade name Polymer JR 4009 from Amerchol, cationic starches, copolymers of diallylammonium salts and acrylamides, quaternized vinyl pyrrolidone/vinyl imidazole polymers, such as, e.g., Luviquat® (BASF), condensation products of polyglycols with amines, quaternized collagen polypeptides, such as for example, lauryldimonium hydroxypropyl hydrolyzed collagen (Lamequat®Grunau), quaternized wheat polypeptides, polyethylene imines, cationic silicone polymers, such as amidomethicone, copolymers of adipic acid and dimethylaminohydroxypropyldiethylene triamine (Cartaretine®/Sandoz), copolymers of acrylic acid and dimethyldiallylammonium chloride (Merquat® 550/Chemviron), polyaminopolyamides, such as, e.g., described in FR 2252840 A as well as their crosslinked water-soluble polymers, cationic chitin derivatives such as, e.g., quaternized chitosan, optionally microcrystallinically dispersed, condensation products of dihaloalkylenes, such as, e.g., dibromobutane with bis-dialkylamines, such as, e.g., bis-dimethylamino-1,3-propane, cationic guar gum, such as, e.g., Jaguar® CBS, Jaguar® C-17, Jaguar® C-16 from Celanese company, quaternized ammonium polymers, such as, e.g., Mirapol® A-15, Mirapol® AD-1, Mirapol® AZ-1 from the Miranol company.

The cationic polymers are preferably added to the inventive compositions in quantities of 0.1 to 10 wt. %, particularly in a quantity of 0.2 to 2 wt. %, in each case based on the total weight of the compositions.

According to the invention, preferred surfactants of the type quaternary ammonium compounds, esterquats and the amidoamines can be added as the cationic surfactants. Preferred quaternary ammonium compounds are ammonium halides, particularly chlorides and bromides, such as alkyltrimethylammonium chlorides, dialkyldimethylammonium chlorides and trialkylmethylammonium chloride, e.g., cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known underthe INCI designations Quaternium-27 and Quaternium-83. The long alkyl chains of the abovementioned surfactants have preferably 10 to 18 carbon atoms.

Esterquats are known compounds, which both comprise at least one esterfunction and also a quaternary ammonium group as structural elements. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines. Such products are marketed, for example, under the trade names Stepantex®, Dehyquart® and Armocare®. The products Armocare® VGH-70, an N,N-bis(2-palmitoyloxyethyl)dimethylammonium chloride, as well as Dehyquart® F-75, Dehyquart®C-4046, Dehyquart® L80 and Dehyquart®AU35 are examples of such esterquats.

The alkylamidoamines are normally manufactured by the amidation of natural or synthetic fatty acids and fatty acid fractions with dialkylaminoamines. According to the invention, a particularly suitable compound from this substance group is represented by stearamidopropyldimethylamine, commercially available under the designation Tegamid® S 18.

The agents used according to the invention preferably comprise the cationic surfactants in quantities of 0.05 to 2 wt. %, based on the total agent. Quantities of 0.1 to 1 wt. % are particularly preferred.

In a further embodiment of the inventive agent, the action can be additionally enhanced by using protein hydrolyzates and their cationic derivatives. Protein hydrolyzates are product mixtures obtained by acid-, base- or enzyme-catalyzed degradation of proteins (albumins).

According to the invention, the added protein hydrolyzates can be of both vegetal as well as of animal origin.

Animal protein hydrolyzates are, for example, elastin, collagen, keratin, milk protein, and silk protein hydrolyzates, which can also be present in the form of their salts. Such products are marketed, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

According to the invention, it is preferred to use protein hydrolyzates of vegetal origin, e.g., soya, almond, rice, pea, potato and wheat protein hydrolyzates. Such products are available, for example, under the trade names Gluadin® (Cognis), DiaMin® (Diamalt), Lexein® (Inolex) and Crotein® (Croda).

Although it is preferred to add the protein hydrolyzates as such, optionally other mixtures containing amino acid or individual amino acids can also be added in their place, such as arginine, lysine, histidine or pyrroglutamic acid. Likewise, it is possible to add derivatives of protein hydrolyzates, e.g., in the form of their fatty acid condensation products. Such products are marketed, for example, under the trade names Lamepon® (Cognis), Gluadin® (Cognis), Lekein® (Inolex), Crolastin® (Croda) or Crotein® (Croda).

According to the invention, the protein hydrolyzates are preferably added in quantities of 0.1 to 6 wt. %, particularly in a quantity of 0.2 to 2 wt. %, in each case based on the total weight of the agent.

Among the preferred vitamins and vitamin derivatives according to the invention are preferred such vitamins, provitamins and vitamin precursors, which are normally classified in the groups A, B, C, E, F and H.

In the group of substances designated as vitamin A, belong retinol (vitamin A₁) as well as 3,4-didehydroretinol (vitamin A₂). β-carotene is the provitamin of retinol. Examples of suitable vitamin A components according to the invention are vitamin A acid and its esters, vitamin A aldehyde and vitamin A alcohol as well as its esters such as the palmitate and acetate. The preparations according to the invention preferably comprise the vitamin A components in amounts of 0.05-1 wt. % based on the total preparation.

The vitamin B group or the vitamin B complex include inter alia
Vitamin B₁ (Thiamine)
Vitamin B₂ (Riboflavine)
Vitamin B₃. The compounds nicotinic acid and nicotinamide are often included under this designation. According to the invention, nicotinamide is preferred and is comprised in the agents according to the invention in amounts of 0.05 to 1 wt. % based on the total agent.
Vitamin B₅ (pantothenic acid and panthenol). In the context of this group, panthenol is preferably used. Usable derivatives of panthenol according to the invention are especially the esters and ethers of panthenol as well as cationically derivatized panthenols. Specific representatives are for example, panthenol triacetate, panthenol monoethyl ether and its monoacetate as well as the cationic panthenol derivatives disclosed in WO 92/13829. The cited compounds of the vitamin B₅ type are comprised in the agents used according to the invention in amounts of 0.005-10 wt. %, based on the total agent. Quantities of 0.1 to 5 wt. % are particularly preferred.
Vitamin B₆ (pyridoxine as well as pyridoxamine and pyridoxal).

Vitamin C (ascorbic acid). Vitamin C is preferably added to the agents according to the invention in amounts of 0.1 to 3 wt. %, based on the total agent. Its use in the form of the palmitate esters can be preferred. Its use in combination with tocopherols can also be preferred.

Vitamin E (Tocopherols, especially α-tocopherol). Tocopherol and its derivatives, among which particularly the esters such as the acetate, the nicotinate, the phosphate and the succinate, are used in the agents according to the invention, preferably comprised in amounts of 0.05-1 wt. %, based on the total agent.

Vitamin F. The term “vitamin F” is usually taken to mean essential fatty acids, particularly linoleic acid, linolenic acid and arachidonoic acid.

Vitamin H. The compound (3aS,4S, 6aR)-2-oxohexahydrothienol[3,4-d]-imidazole-4-valeric acid denotes Vitamin H, for which the trivial name biotin has become accepted. The agents according to the invention preferably comprise biotin in amounts of 0.0001 to 1.0 wt. %, particularly in amounts of 0.001 to 0.01 wt. %.

The agents according to the invention preferably comprise vitamins, provitamins and vitamin precursors from groups A, B, E and H.

Panthenol, and its derivatives as well as nicotinamide and biotin are particularly preferred active care products; panthenol and its derivatives are especially preferred.

The UV-filters used in the context of the invention are chosen from substituted benzophenones, p-aminobenzoates, diphenylacrylates, cinnamates, salicylates, benzimidazoles and o-aminobenzoates.

Particularly preferred UV-filters according to the invention are 4-amino-benzoic acid, N,N,N-trimethyl-4-(2-oxoborn-3-ylidenemethyl)aniline methylsulfate, 3,3,5-trimethylcyclohexyl salicylate (Homosalate), 2-hydroxy-4-methoxy-benzophenone (Benzophenone-3; Uvinul® M 40, Uvasorb® MET, Neo Heliopan® BB, Eusolex®4360), 2-phenylbenzimidazol-5-sulfonic acid and their potassium, sodium und triethanolamine salts (phenylbenzimidazole sulfonic acid; Parsol® HS; Neo Heliopan® Hydro), 3,3′-(1,4-phenylenedimethylene)-bis(7,7-dimethyl-2-oxo-bicyclo-[2.2.1]hept-1-yl-methanesulfonic acid) and their salts, 1-(4-tert.-butylphenyl)-3-(4-methoxyphenyl)propane-1,3-dione (butylmethoxydibenzoylmethane; Parsol® 1789, Eusolex® 9020), a-(2-oxoborn-3-ylidene)-toluene-4-sulfonic acid and salts thereof, ethoxylated ethyl 4-aminobenzoate (PEG-25 PABA; Uvinul® P 25), 2-ethylhexyl 4-dimethylaminobenzoate (Octyl Dimethyl PABA; Uvasorb® DMO, Escalol® 507, Eusolex® 6007), 2-ethylhexyl salicylate (Octyl Salicylate; Escalol® 587, Neo Heliopan® OS, Uvinul® O18), isopentyl 4-methoxycinnamate (isoamyl p-methoxycinnamate; Neo Heliopan® E 1000), 2-ethylhexyl 4-methoxycinnamate (Octyl Methoxycinnamate; Parsol® MCX, Escalol® 557, Neo Heliopan® AV), 2-hydroxy-4-methoxybenzophenone-5-sulfonic acid and sodium salts thereof, (benzophenone-4; Uvinul® MS 40; Uvasorb® S 5), 3-(4′-methylbenzylidene)-D,L-camphor (4-methylbenzyldene camphor; Parsol® 5000, Eusolex® 6300), 3benzylidene-camphor (3-Benzylidene camphor), 4-isopropylbenzyl salicylate, 2,4,6-trianilino-(p-carbo-2′-ethylhexyl-1′-oxy)-1,3,5-triazine, 3-imidazol-4-yl-acrylic acid and its ethyl ester, polymers of N (2 and 4)-[2-oxoborn-3-ylidenemethyl]benzylacrylamide, 2,4-dihydroxybenzophenone (Benzophenone-1; Uvasorb®20H, Uvinul®400), 2-ethylhexyl ester of 1,1′-diphenylacrylonitrilic acid (Octocrylene; Eusolex® OCR, Neo Heliopan® Type 303, Uvinul® N 539 SG), menthyl o-aminobenzoate (Menthyl Anthranilate; Neo Heliopan® MA), 2,2′,4,4′-tetrahydroxybenzophenone (Benzophenone-2, Uvinul® D-50), 2,2′-dihydroxy-4,4′-dimethoxybenzophenone (Benzophenone-6), 2,2′-dihydroxy-4,4′-dimethoxybenzophenone-5-sodium sulfonate and 2′-ethylhexyl 2-cyano-3,3-diphenylacrylate.

According to the invention, benzophenone-3 is particularly preferred.

An example of a particularly preferred, water-soluble UV-filter according to the invention is 2-hydroxy-4-methoxybenzophenone-5-sulfonic acid (Uvasorb® S 5, Uvinul® MS 40, Escalol® 577; benzophenone-4).

According to the invention, the UV-filters are preferably added in quantities of 0.01 to 30 wt. %, particularly in quantities of 0,05 to 5 wt. %, in each case based on the total weight of the agent.

Suitable color pigments are for example the pigments with the C. I. names Pigment Red 57:1, Pigment Red 57:2, Pigment Red 172, Pigment Red 90:1, Pigment Yellow 100, Pigment Yellow 115, Pigment Red 174, Pigment Red 4, Pigment Blue 29, Pigment Violet 15, Pigment Violet 16, Pigment Red 29, Pigment Green 17, Pigment Green 18, Natural Red 4, Pigment White 6, Pigment White 14 and Pigment White 31.

According to the invention, all waxes that have a melting point in the range 40 to 90° C. can be used as oil-soluble waxes. In the context of the invention, all materials can be used that are listed in, e.g., Ullmanns Enzyklopädie der technischen Chemie, 4^{th} Edition, Volume 24, page 3, left column and which are physiologically compatible.

The waxes are preferably selected among vegetal, animal and mineral waxes, preferred waxes being those with a melting point in the range 50 to 85° C., particularly from 60 to 75° C.

According to the invention, preferred waxes are beeswax (Cera Alba), carnauba wax, candelilla wax, montan wax, microcrystalline waxes (microcrystalline paraffins), cetyl palmitate, or mixtures of these waxes.

Preferred wax mixtures in the context of the invention are, for example, those commercially available under the designations “Special Wax 76860E” (mixture of cetyl palmitate, beeswax, microcrystalline wax and polyethylene with a melting range of 73 to 75° C.—manufacturer: Kahl & Co.), Polywax® GP 200 (mixture of stearyl alcohol and polyethylene glcol stearate with a melting point of 47 to 51° C.—manufacturer: Croda) and “Weichceresin® FL 400” (a vaseline/paraffin oil/wax mixture with a melting point of 50 to 54° C.—manufacturer: Parafluid Mineralolgesellschaft).

Liquid waxes, such as for example, jojoba oil, can also be used according to the invention.

With regard to further standard ingredients, reference is expressly made to monographs known to the expert, for example K. Schrader, Grundlagen und Rezepturen der Kosmetika, Dr. Alfred Huthig Verlag, Heidelberg.

Packaging of the agents according to the invention is not subject to any restrictions. They can be packaged as lotions, pumpable hair sprays, pumpable spray settings, care sprays, no-curl sprays, anti-tangling sprays or gloss sprays.

A second subject of the invention is a process for styling hair, wherein a agent according to the invention is applied to the hair, the hair is shaped and the hair is finally dried with hot air or in air.

A third subject of the invention is the use of the inventive agent for styling hair.

A fourth subject of the invention is the use of the inventive agent for producing glossy hair.

The following examples are intended to describe the subjects of the invention in more detail, without limiting them in any way.

The present invention is further illustrated by the following Examples:

### EXAMPLES

Gloss Spray:

The following quantities refer to wt. %.

| | |
|---|---|
| In water | 49.65, |

heated to ca. 65° C., was added with stirring

| | |
|---|---|
| PVP¹ | 5.00 |

¹Luviskol ® K 30 powder (INCI: Polyvinyl pyrrolidone; 95-100% AS)

and dissolved to a clear solution.

The following were added one after the other with stirring and without heating

| | |
|---|---|
| Panthenol | 0.20 |
| Dimethicone | 4.00 |
| Trisiloxane | 11.00 |
| Essential Oil | 0.15 |
| Triethyl citrate | 29.00 |
| Isoamyl p-methoxycinnamate² | 1.00 |

²Neoheliopan ® E 1000 (INCI:

[symbol]

1. A sprayable hair treatment agent comprising: (A) an aqueous phase, (B) an oil phase and (C) a phase comprising a trifunctional ester wherein the composition is a stable, three-phase system.

2. The agent of claim 1 wherein the ester is composed of units a) and b) wherein unit a) is an alcohol or a carboxylic acid having chain lengths of from 2 to 10 carbon atoms and unit b) is a compound of Formula (I), 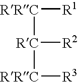
wherein each of R¹, R² and R³ is independently an —OH or —COOH group and each of R, R″ is independently —H, —OH or a Ci-C4-alkyl group.

3. The agent of claim 1 wherein each of R¹, R² and R³ is —OH.

4. The agent of claim 1 wherein the compound of Formula (I) is glycerol.

5. The agent of claim 1 wherein the ester is composed of glycerol and a carboxylic acid having from 2 to 6 carbon atoms.

6. The agent of claim 1 wherein the ester is glycerol triacetate.

7. The agent of claim 1 wherein each of R¹, R² and R³ is a —COOH group.

8. The agent of claim 1 wherein the compound of Formula (I) is citric acid.

9. The agent of claim 1 wherein the ester is composed of citric acid and an alcohol having from 2 to 6 carbon atoms.

10. The agent of claim 1 wherein the ester is triethyl citrate.

11. The agent of claim 1 wherein the amount of the ester phase is from 2 to 98 wt. %, based on the total weight of the agent.

12. The agent of claim 1 wherein the oil phase is vegetal, mineral oils, synthetic oils and mixtures thereof.

13. The agent of claim 1 wherein the oil phase is a liquid, halogenated hydrocarbon.

14. The agent of claim 13 wherein the liquid, halogenated hydrocarbon is ethyl perfluorobutyl ether, perfluorodecahydronaphthalene and mixtures thereof.

15. The agent of claim 1 wherein the amount of phase (B) is from 2 to 98 wt. %, based on the total weight of the agent.

16. The agent of claim 1 wherein the amount of phase (A) is from 2 to 98 wt. %, based on the total weight of the agent.

17. The agent of claim 1 further comprising water-soluble film builders.

18. The agent of claim 1 further comprising an oil or water-soluble care substance, a UV-absorber, a perfume oil, a colorant and mixtures thereof.

19. The agent of claim 18 wherein the water-soluble care substance is panthenol.

20. A process for styling hair comprising the steps of: (1) contacting hair with the agent of claim 1; (2) shaping the treated hair from step (1); (3) drying the shaped hair from step (2).
